(19) United States
(12) Patent Application Publication
(10) Pub. No.: US 2019/0206575 A1
(43) Pub. Date: Jul. 4, 2019

(54) SMART CLUSTERING AND CLUSTER UPDATING

(71) Applicant: EasyMarkit Software Inc., Vancouver (CA)

(72) Inventors: Andrew Batey, Vancouver (CA); Owen Ingraham, Vancouver (CA)

(73) Assignee: EasyMarkit Software Inc., Vancouver (CA)

(21) Appl. No.: 15/981,036

(22) Filed: May 16, 2018

Related U.S. Application Data
(63) Continuation-in-part of application No. 15/862,055, filed on Jan. 4, 2018.

(51) Int. Cl. G16H 70/20; G06F 17/30; G06K 9/62; G06F 17/18

(52) U.S. Cl. CPC G16H 70/20; G06F 17/30707; G06F 17/18; G06K 9/6223; G06K 9/6262

(57) ABSTRACT

Described herein are techniques and mechanisms for medical practice data analytics. According to various embodiments, a system may include a clinic information database, a clinic data cluster engine, and a clinic data analytics engine. The clinic information database may store clinic data characterizing each of a plurality of medical practice clinics. The clinic data cluster engine may determine a respective plurality of clinic clusters based on the clinic information for each of a plurality of clustering mechanisms. The clinic data analytics engine may evaluate the performance of each of the plurality of clustering mechanisms to produce a respective performance evaluation by determining a respective predicted outcome variable for each of the respective clustering mechanisms and each of the respective clinic clusters and comparing each of the respective predicted outcome variable with a respective observed outcome variable.

### CROSS-REFERENCE TO RELATED APPLICATIONS

The present application is a continuation-in-part of and claims priority under 35 U.S.C. 120 to U.S. patent application Ser. No. 15/862,055 (Attorney Docket No. EASMP003), filed Jan. 4, 2018, by Andrew Batey and Owen Ingraham, titled “Data Integration and Enrichment”, which is hereby incorporated by reference in its entirety and in all purposes.

### TECHNICAL FIELD

The present disclosure relates to the collection, aggregation, supplementation, clustering, and analysis of data associated with medical practices.

### DESCRIPTION OF RELATED ART

Managing a modern medical practice requires overcoming significant challenges in the area of information technology. Patient data is increasingly stored and managed in digital rather than paper records. However, many jurisdictions substantially restrict the sharing and distribution of medical records in an effort to protect patient privacy. In addition, security concerns are paramount when dealing with patient medical record data, since a data breach could reveal sensitive information for thousands or millions of patients. Complicating matters further is the fact that investigating, implementing, and maintaining complex information technology is outside the area of expertise of most medical practitioners, assistants, and administrators.

One area of technology that presents particular information technology challenges to a modern medical practice is data analytics. Medical practices collect a substantial amount of data, including data related to patient demographics, billing practices, appointment scheduling, and many other information domains. Such data could in theory be used to improve the efficiency of operations and provide more effective medical services. However, how best to utilize the data is unclear. Accordingly, improved techniques and mechanisms for aggregating, analyzing, processing, and acting upon medical practice data are desired.

### SUMMARY

The following presents a simplified summary of the disclosure in order to provide a basic understanding of certain embodiments of the invention. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

In general, certain embodiments of the present invention provide mechanisms, techniques, and computer readable media having instructions stored thereon for medical practice data analytics. According to various embodiments, a system may include a clinic information database, a clinic data cluster engine, and a clinic data analytics engine. The clinic information database may store clinic data characterizing each of a plurality of medical practice clinics. The clinic data cluster engine may determine a respective plurality of clinic clusters based on the clinic information for each of a plurality of clustering mechanisms. The clinic data analytics engine may evaluate the performance of each of the plurality of clustering mechanisms to produce a respective performance evaluation by determining a respective predicted outcome variable for each of the respective clustering mechanisms and each of the respective clinic clusters and comparing each of the respective predicted outcome variable with a respective observed outcome variable.

According to various embodiments, the cluster data analytics engine may be further configured to select a designated one of the clustering mechanisms based on the performance evaluations and to apply the designated clustering mechanism to predict a designated outcome variable for a designated one of the medical practice clinics. In particular embodiments, applying the designated clustering mechanism involves identifying an actual value for the designated outcome variable for the designated medical practice clinic, determining a proposed policy change for the medical practice clinic, and predicting a second value for the designated outcome variable based on the proposed policy change. In some implementations, the system may electronically transmit an indication of the proposed policy change and the predicted second value for the designated outcome variable to a computing device associated with the medical practice clinic.

### BRIEF DESCRIPTION OF THE DRAWINGS

The disclosure may best be understood by reference to the following description taken in conjunction with the accompanying drawings, which illustrate particular embodiments.

FIG. 1 illustrates an example of an overview method for medical practice clinic data analytics that can be performed in conjunction with various techniques and mechanisms of the present invention.

FIG. 2 illustrates an example of a system that may be used to perform clinic clustering operations in conjunction with various techniques and mechanisms of the present invention.

FIG. 3 illustrates one example of a data retrieval method performed in accordance with one or more embodiments.

FIG. 4 illustrates one example of a system.

FIG. 5 illustrates one example of a method for assigning one or more clinics to profile clusters.

FIG. 6 illustrates one example of an arrangement of medical clinics into clusters.

FIG. 7 illustrates one example of a medical practice analytics method that may be performed in accordance with techniques and mechanisms described herein.

FIG. 8 illustrates one example of a clustering regression testing method that may be performed in accordance with techniques and mechanisms described herein.

FIG. 9 illustrates one example of a prediction regression testing method that may be performed in accordance with techniques and mechanisms described herein.

FIG. 10 illustrates one example of a performance engine analytics method 1000 that may be performed in accordance with techniques and mechanisms described herein.

### DESCRIPTION OF EXAMPLE EMBODIMENTS

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

For example, the techniques of the present invention will be described in the context of data associated with dental or medical practices. However, it should be noted that the techniques of the present invention apply to a wide variety of different service industries and data sources. In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present invention unless otherwise noted. Furthermore, the techniques and mechanisms of the present invention will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

Overview

According to various embodiments, techniques and mechanisms described herein facilitate the collection, aggregation, supplementation, and analysis of medical practice clinic data. Clinic data stored on disparate and proprietary information technology systems at medical practice clinics may be received via customized application procedure interfaces. The resulting data may then be supplemented with data received from external sources. After aggregation, the clinic-level data may be used to organize clinics into clusters. Each clinic may then be provided with data analytics information reflecting a comparison of the clinic to other clinics within the same cluster.

### Example Embodiments

Medical practices collect a substantial amount of data, including data related to patient demographics, billing practices, appointment scheduling, and many other information domains. Such data could in theory be used to improve the efficiency of operations and provide more effective medical services. However, the analysis of such data presents numerous technical challenges not addressed by current approaches.

First, the use of such data within an individual clinic is limited because the data cannot be compared to data from other clinics. For example, in order to determine how a clinic is performing along a particular dimension, such as average appointments per practitioner per day, data from the clinic would need to be compared with data associated with other clinics.

Second, the task of collecting data from different clinics presents substantial technical challenge. Medical clinics often employ proprietary and specialized systems to manage the medical practice. Accordingly, data relevant to analyzing medical practices across different clinics is often stored in different formats that are not accessible in a standardized way.

Third, the use of medical practice data is often restricted by privacy regulations. Such regulations are often specific to a particular geographic area such as a state or country and thus can vary from clinic to clinic. Accordingly, conventional data analytics approaches are often inapplicable to medical practices because they do not take into account medical record privacy restrictions.

Fourth, the analysis of medical practice data is made more complex by substantial differences between clinics. Clinics may differ along a number of dimensions such as the type of medical practice, the number of practitioners at the clinic, the demographics of the patient base, and the clinic's membership in an organization or group of clinics. For these reasons, comparing a clinic along a particular dimension (e.g., profitability per practitioner) to all other clinics would produce an estimate that is not particularly helpful because it would not represent a like-to-like comparison.

According to various embodiments, techniques and mechanisms described herein provide technical solutions the address the aforementioned technical problems. For example, a systems architecture includes custom connectors for retrieving clinic data from a variety of clinic information technology systems. In addition, supplemental data may be retrieved from one or more external sources.

According to various embodiments, the collected data may be aggregated and analyzed. For instance, clinics may be divided into clusters. Then, a clinic may be compared with other clinics in the same cluster to determine performance data. The performance data may be used to transmit a medical practice analytics message that indicates a performance characteristic associated with the medical practice.

In particular embodiments, the techniques and mechanisms described herein may provide any or all of several advantages over past approaches. First, a clinic's data may be freed for analysis from the proprietary systems on which it is stored. Second, internal data associated with a medical practice clinic may be supplemented with data from sources other than the medical practice clinic itself. Third, a clinic may be provided with one or more analytics messages that compare the performance of the clinic on one or more dimensions with other clinics having similar characteristics, thus facilitating a like-to-like comparison.

According to various embodiments, the term “medical practice” or “medical clinic” as used herein may apply to any or all of services related to a wide range of health services practices. These include, but are not limited to, dental clinics, veterinary clinics, doctor's clinics, surgical practices, orthodontics practices, orthopedic practices, and physical therapy practices. In particular embodiments, the term “medical practice” or “medical clinic” may refer to any or all of a broad range of health-focused service providers such as psychiatrist, psychologists, massage therapists, physical therapists, occupational therapists, pharmacists, social workers, dermatologists, or dieticians.

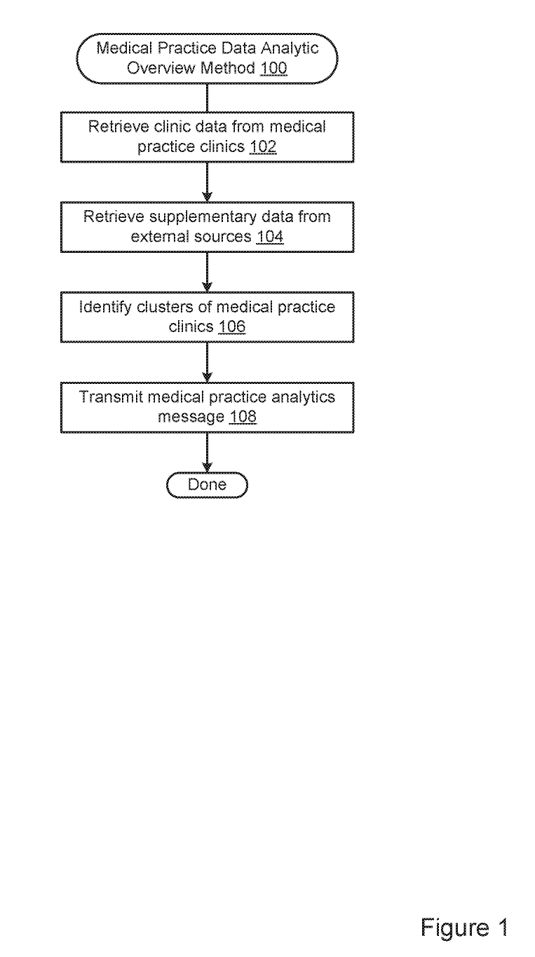

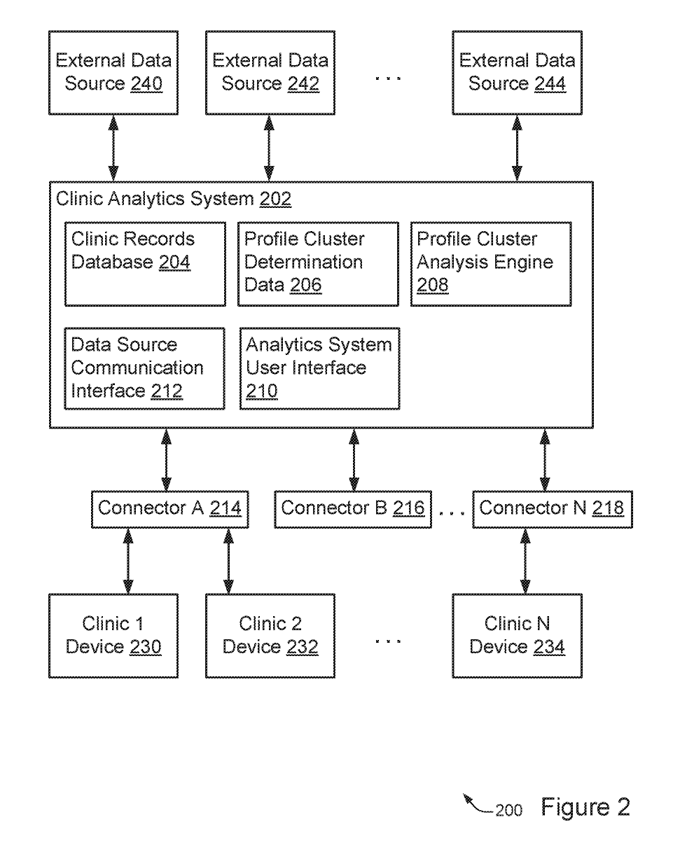

FIG. 1 illustrates an example of an overview method 100 for clinic data analytics that can be performed in conjunction with various techniques and mechanisms of the present invention. According to various embodiments, the method 100 may be performed in order to facilitate more accurate and comprehensive medical practice analytics based on a like-to-like comparison of the medical practice with other similarly situated medical practices. The method 100 may be performed at a clinic data analytic system. An example of a clinic data analytic system is discussed in additional detail with respect to FIG. 2.

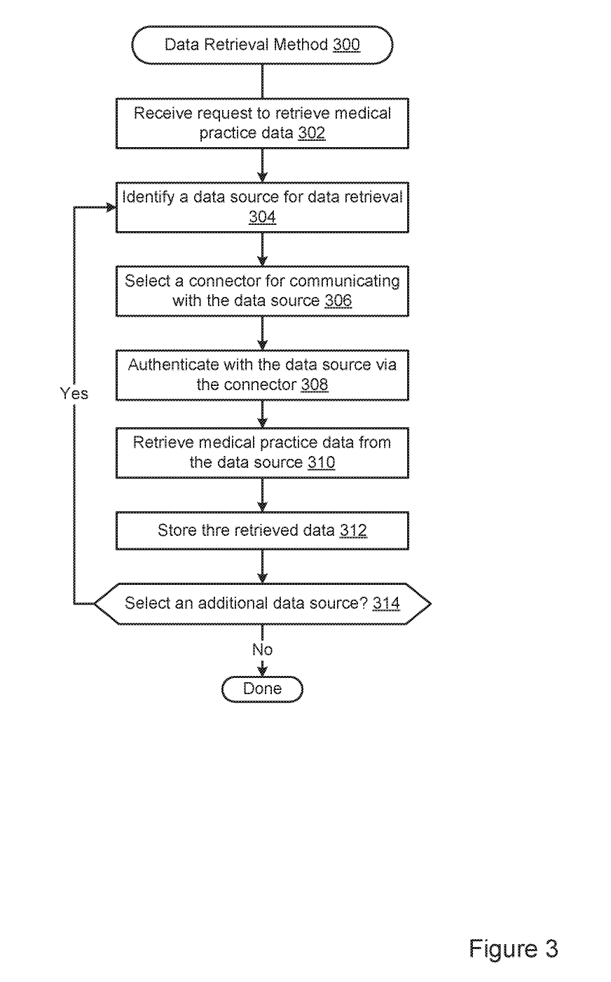

At 102, clinic data is retrieved from a group of medical practice clinics. According to various embodiments, clinic data may be retrieved from a clinic by communicating with an information management system associated with the clinic. Such information may include, but is not limited to: patient demographic data, patient billing data, practitioner data, clinic location data, appointment scheduling data, medical procedure data, and patient interaction data. Patient interaction data may include information such as products or services provided to patients, patient bill payment information, appointment cancellations, appointment no-shows, and/or other aspects of patient behavior. Techniques for retrieving data from a medical practice clinic are discussed in further detail with respect to FIG. 3.

At 104, supplementary data is retrieved from one or more external sources. In some embodiments, such information may be retrieved from one or more public or private information sources accessible via network communications. As a first example, demographic data associated with a particular location may be retrieved. As a second example, billing practices data may be retrieved from medical insurers. As a third example, government guidelines may be retrieved that indicate standards of care such as the type and frequency of particular medical treatments. As a fourth example, social media or other profile data may be retrieved from websites such as WebMD, medical associations, LinkedIn, Facebook, or a medical practice's own websites. Such information may include the number, ages, medical schools, and other such data about doctors that may assist in the development of accurate profiles. For instance, older doctors may be more interested in profiting from a business rather than growing a patient base. As a fifth example, data may be retrieved from a third-party enrichment service such as ClearBit. As a sixth example, data may be provided to the system manually. For instance, data may be retrieved from a public library or from the results of a survey of medical practice clinics which may indicate information such as the software in use by the medical practice clinics and the preferences of medical practice clinic managers.

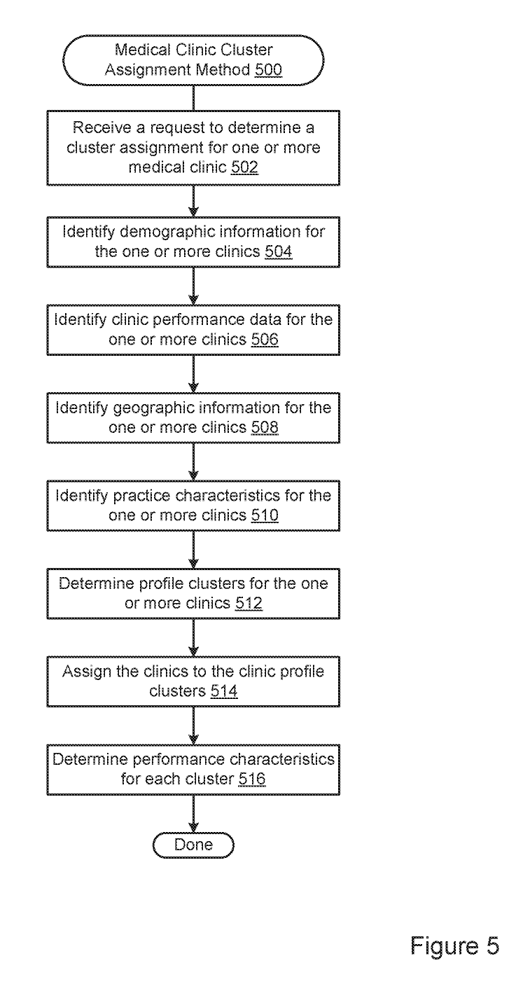

At 106, clusters of medical practice clinics are identified. According to various embodiments, the clusters may be identified by dividing medical practice clinics into groups of similar characteristics based on observed characteristics. For example, clinics may be clustered based on having similar numbers of practitioners, patient demographic characteristics, and/or geographic location. Techniques for clustering medical practice clinics are discussed in additional detail with respect to FIG. 5.

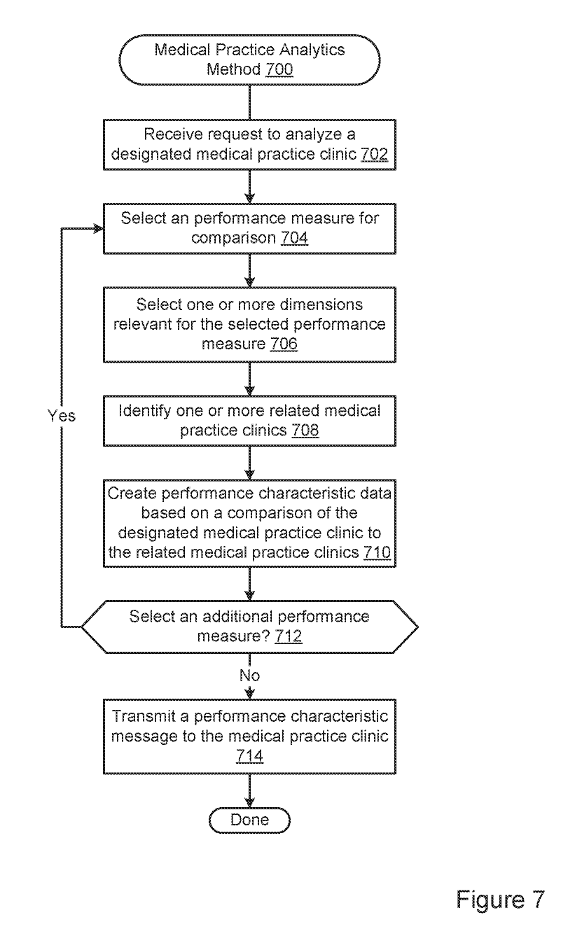

At 108, a medical practice analytics message is transmitted to a medical practice clinic. According to various embodiments, transmitting the medical practice analytics information may involve determining a cluster associated with the medical practice clinic, comparing data associated with the clinic with other clinics in the same cluster, and determining a performance characteristic based on the comparison. Techniques for transmitting a medical practice analytics message are discussed in further detail with respect to FIG. 7.

The system 200 includes a patient clinic analytics system 202 in communication with devices 230 232, and 234 associated with medical practice clinics. The clinic analytics system 202 also includes a clinic records database 204, profile cluster determination data 206, a profile cluster analysis engine 208, an analytics system user interface 210, and a data source communication interface 212. The patient clinic analytics system 202 is also in communication with external data sources 240, 242, and 244.

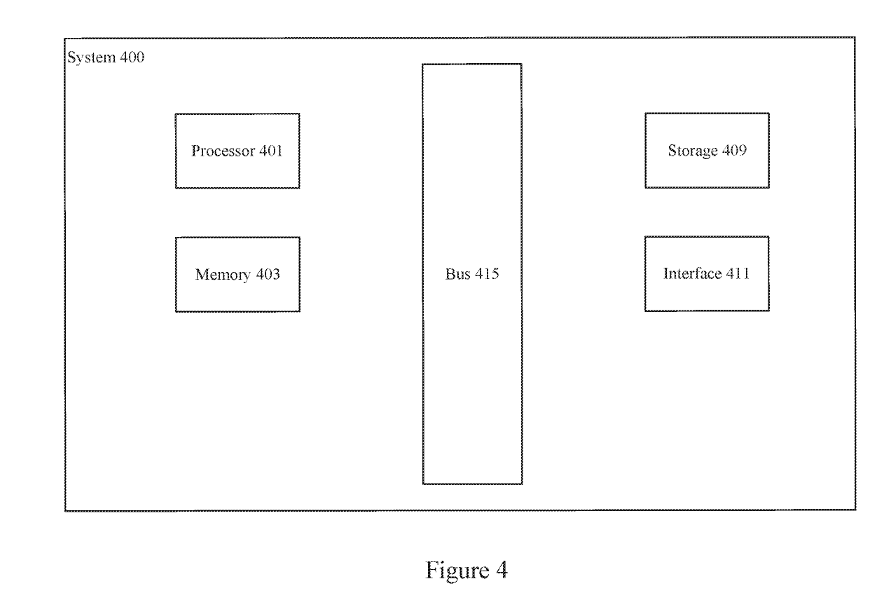

In some embodiments, the clinic analytics system 202 may be implemented on a server such as the system 400 shown in FIG. 4. Alternately, different portions of the clinic analytics system may be implemented on different computing devices. In some configurations, the clinic analytics system 202 may be implemented via a cloud computing architecture.

According to various embodiments, the clinic records database 204 includes information about medical practice clinics registered with the clinic analytics system. For example, the clinic records database 204 may include any or all of demographic information, insurance information, past and future appointment scheduling information, medical record information, and other such data associated with individual patients. As another example, the clinic records database 204 may store practice information such as practitioner names, practitioner practice areas, practitioner types, and other such information. As yet another example, the clinic records database 204 may store clinic information such as geographic location data, co-ownership data, account information, or other such data.

In some implementations, the profile cluster determination data 206 includes any information suitable for determining clusters of medical clinics. The profile cluster determination data 206 may include at least a portion of the information stored in patient records database 204, such as patient demographic information and medical practice information. The profile cluster determination data 206 may also include other information, such as information collected from one or more external data sources.

According to various embodiments, the profile cluster analysis engine 208 may process the profile cluster determination data 206 to determine clusters of clinics. The clustering process may involve identifying groups of clinics that share similar profile cluster determination data. By clustering clinics that share similar data, clinics may be compared along one or more performance dimensions in a like-to-like comparison. Techniques for clustering medical practice clinics are discussed in additional detail with respect to FIG. 5.

In some implementations, the clinic analytics system 202 may be accessed via the analytics system user interface 210. The analytics system user interface 210 may be implemented as, for example, a user interface presented in a website or an application installed on a computing device. The user interface 210 may support operations such as user authentication, clinic data access configuration, connector configuration, clinic clustering configuration, and clinic data analytics configuration. The user interface 210 may be accessed by any of a variety of users such as systems administrators or individuals associated with one or more medical practice clinics.

According to various embodiments, the data source communication interface 212 is configured to facilitate communications between the clinic analytics system 202 and external data sources and clinic devices via a network such as the internet. For example, the data source communications interface 212 may retrieve information from a clinic device via one of the connectors 214, 216, and 218. As another example, the data source communications interface 212 may retrieve information from an external data source such as the data sources 240, 242, and 244.

According to various embodiments, each clinic device 230, 232, and 234 may be associated with a respective medical practice clinic. For example, a clinic device may be a computing system that manages practice data associated with the clinic. A clinic device may be located at the physical premises of the clinic or may be located outside the clinic, such as in a cloud computing environment.

Each clinic device may be configured to communicate via a respective application procedure interface. For example, data may be stored at the clinic device in a proprietary data storage system associated with a proprietary clinic data management system having a proprietary interface. The data may be retrieved via a network by transmitting and receiving messages as specified by the proprietary interface. Examples of such data management systems include, but are not limited to: Dentrix, Eaglesoft, ClearDent, and PracticeX. As another example, data may be stored at the clinic device in a proprietary data storage system associated with a proprietary patient communication system having a proprietary interface. The data may be retrieved via a network by transmitting and receiving messages as specified by the proprietary interface. Examples of such patient communications systems include, but are not limited to: DemandForce, SolutionReach, and Lighthouse360.

In particular embodiments, the data source communications interface 212 can retrieve data from a proprietary clinic data management system via a connector, the clinic analytics system 202 may be used to provide data analytics services distinct from both a clinic data management system and a patient communication system.

According to various embodiments, the data source communication interface 212 may communicate with a clinic device via a connector such as the connector A 214, the connector B 216, or the connector N 218. Each connector may be configured to facilitate communications between a proprietary information storage system at a clinic device and the data source communication interface 212.

In some embodiments, the data source communication interface 212 may send a message such as a request to retrieve information in a standardized format to the appropriate connector. The connector may then translate the message to formulate an application procedure call appropriate to the clinic device associated with the connector. Next, the connector may transmit the application procedure call to the clinic device and receive a response via the proprietary application procedure interface. Finally, the connector may translate the proprietary application procedure interface response to a standardized response and provide the standardized response to the data source communication interface 212.

In particular embodiments, more than one clinic device may share a common connector. For example, the clinic devices 1 230 and 2 232 shown in FIG. 2 share the connector A 214. Such a configuration may occur if, for example, the clinic devices have the same proprietary clinic data management system.

In some implementations, the clinic analytics system 202 may retrieve information from one or more external data sources such as the external data sources 240, 242, and 244 shown in FIG. 2. As used herein, the term “external data source” refers to any data source not directly associated with a medical practice clinic.

In particular embodiments, the clinics analytics system 202 may communicate with an external data source via a connector such as the connector 1 252, the connector 2 254, or the connector k 256. Each connector may be configured to perform one or more application procedure interface calls to retrieve data from an external source. Then, the data may be provided for use by the clinic analytics system 202 in a standardized fashion.

According to various embodiments, an external data source may be employed to retrieve data to supplement data retrieved directly from a clinic. For example, a geographic location associated with a clinic may be used to query an external data source for information about that geographic location. Such information may include, but is not limited to: income data, population density data, commuting distance data, demographic data, or other information associated with the geographic location or people living in the geographic location.

In particular embodiments, any of a variety of external data sources may be used. Examples of external data sources include, but are not limited to: publicly available search engines, publicly available data repositories, privately available search engines, and privately available data repositories. In some instances, an external data source may be designed for information access and retrieval. Alternately, an external data source may be a public-facing website that is scraped to retrieve the appropriate data.

In particular embodiments, one or more of the components illustrated in FIG. 2 may be omitted. For example, the clinic analytics system 202 may communication directly with one or more of the external data sources and/or one or more of the clinic devices directly without the aid of a connector.

FIG. 3 illustrates one example of a data retrieval method 300 performed in accordance with one or more embodiments. According to various embodiments, the method 300 may be used to retrieve data used in the performance of clinic analytics. The method 300 may be performed at a clinic data analytics system such as the system 200 shown in FIG. 2.

At 302, a request to retrieve medical practice data is received. According to various embodiments, the request may be generated when any of various conditions are met. For example, the request may be generated periodically, such as daily, hourly, or weekly. As another example, the request may be generated when an event occurs, such as when a new clinic or data source is connected with the system. As yet another example, the request may be generated when triggered by a user such as a systems administrator.

At 304, a data source for data retrieval is identified. According to various embodiments, the identified data source may be an information system associated with a clinic or may be an external data source, as discussed with respect to FIG. 2. The data source may be selected based on any of various criteria. For example, data may be retrieved from a data source periodically, upon request, or when it is determined that updated data is available from the data source.

At 306, a connector for communicating with the data source is selected. As discussed with respect to FIG. 2, many data sources involve proprietary data storage systems that are not accessible via standard application procedure interfaces. Accordingly, the system may evaluate a data source to determine an appropriate connector. For example, a systems administrator may establish a configuration parameter that links a data source with a particular connector. As another example, the clinic analytics system may communicate with the data source to determine which connector is suitable. As yet another example, the clinic analytics system may try to communicate with the data source via different connectors until a suitable connector is found.

At 308, the analytics system authenticates with the data source via the connector. According to various embodiments, authenticating with the data source may involve operations such as identifying credentials, transmitting the credentials to the data source, and establishing a communications session between the data source and the analytics system. In particular embodiments, the analytics system need not authenticate with one or more data sources. For example, some external data sources may be made publicly available.

At 310, medical practice data is retrieved from the data source. According to various embodiments, the medical practice data may include any suitable information for conducting medical practice analytics. For example, the medical practice data may include clinic data such as patient demographics, clinic performance information, geographic location data, or clinic characteristics. As another example, the medical practice data may include supplemental data such as data about the geographic location in which the clinic is situated.

At 312, the retrieved medical practice data is stored. As discussed with respect to FIG. 2, the medical practice data may be stored in a data store such as a clinic records database. The database may include information retrieved directly from clinics as well as supplemental information retrieved from one or more external data sources.

At 314, a determination is made as to whether to select an additional data source for data retrieval. According to various embodiments, additional data sources may be selected until data is retrieved from all suitable data sources. As discussed with respect to operation 304, data sources may be identified for data retrieval periodically, upon detection of a triggering event, or upon request.

FIG. 4 illustrates one example of a server. According to particular embodiments, a system 400 suitable for implementing particular embodiments of the present invention includes a processor 401, a memory module 403, a storage device 409, an interface 411, and a bus 414 (e.g., a PCI bus or other interconnection fabric) and operates as a patient clinic analytics system. When acting under the control of appropriate software or firmware, the processor 401 is responsible for performing profile cluster analysis. Various specially configured devices can also be used in place of a processor 401 or in addition to processor 401. The interface 411 is typically configured to send and receive data packets or data segments over a network. The storage device 409 may include one or more of a network attached storage (NAS), a storage area network (SAN) system, a local hard disk, or any other suitable component.

Particular examples of interfaces supported include Ethernet interfaces, frame relay interfaces, cable interfaces, DSL interfaces, token ring interfaces, and the like. In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control communications-intensive tasks such as packet switching.

Although a particular server is described, it should be recognized that a variety of alternative configurations are possible. For example, some modules may be implemented on another device connected to the server. A variety of configurations are possible.

FIG. 5 illustrates one example of a method 500 for assigning one or more clinics to profile clusters, performed in accordance with one or more embodiments. one example of a method 500 for assigning one or more clinics to profile clusters. According to various embodiments, the method 500 may be implemented on a profile cluster analysis engine in a patient clinic analytics system, such as the profile cluster analysis engine 208 shown in FIG. 2.

At 502, a request to determine a cluster assignment for one or more medical clinics is received. According to various embodiments, a cluster or clusters may be determined at any or all of various points in time. For example, clusters may be determined when the system is initialized with clinic data for the first time. As another example, new clinics may be assigned to clusters when they are entered into the system. As still another example, clinic assignments may be periodically reevaluated to reflect new data. For instance, clusters may be reevaluated once per hour, once per day, once per week, once per month, or when a designated number of changes to clinic data have been detected.

At 504, demographic information for the one or more clinics is identified. In some implementations, demographic data may include any information characterizing the attributes of patients of the medical practice. This information may include, but is not limited to: age, sex, race, medical history, profession, employer, marital status, insurance provider, income level, residence location, and parental status. Such information may be collected when a patient is onboarded at as a new patient at a medical practice for the first time and may be updated periodically, for instance upon each appointment.

At 506, clinic performance data for the one or more clinics is identified. According to various embodiments, clinic performance data may include any information associated with the medical and/or economic performance of the medical practice associated with the clinic. For example, the clinic performance data may include billing information for different procedures, collection information for bills sent to patients, efficiency information such as procedures per practitioner per day, amount spent on staff or overhead, or other such characteristics.

At 508, geographic information is identified for the one or more clinics. According to various embodiments, the geographic information may indicate a city, state, country, zip code, address, or other location information associated with the clinic. In particular embodiments, the geographic information may include metadata associated with a specific locale. For example, the geographic information may indicate a regulatory regime governing medical practices in the geographic area. As another example, the geographic information may include demographic information associated with the geographic area such as income data, population density data, occupation data, a percentage of people having pre-paid rather than contract phone service, an average distance traveled by patients to reach the clinic, or other such information.

At 510, one or more practice characteristics for the one or more clinics are identified. In some implementations, practice characteristic data may include information about the medical practice associated with the clinic. Medical practice data may include, but is not limited to: the number of medical practitioners, the number of medical assistants, the number of administrators, the number of patients, location, average patient income level, clinic profitability, insurance providers accepted, practice management software (e.g., Dentrix), and information technological characteristics.

In particular embodiments, the number of medical practitioners may be divided by type. For instance, clinic characteristic data may identify a number of doctors, dentists, veterinarians, hygienists, certified dental assistants, nurses, office managers, receptionists, and the like. Such information may be collected when a clinic is added to the system and may be updated periodically, for instance once per month.

According to various embodiments, different types of data may be available for different clinics. For example, practice characteristic data and/or patient demographic data may be available for most or all clinics, even those that are newly added to the system. However, clinic performance data may not be available for some clinics, such as those newly added to the system.

At 512, profile clusters are determined for the one or more clinics. According to various embodiments, clinics may be clustered on any available data. For example, geographic, practitioner, and practice characteristic data may be available for all or virtually all clinics, while patient demographic information may be incomplete for clinics newly added to the system. Thus, clinics may be grouped according to similarity along demographic, practitioner, and practice characteristic dimensions to determine an initial assignment of clusters.

In particular embodiments, clusters may be determined in a hierarchical fashion. For example, a particular cluster of clinics may include medical practices located within the same geographic region. However, this cluster of clinics may include one group that is associated with lower-income patients and another group that is associated with higher-income patients. In this example, the two groups may be treated as sub-clusters of the larger cluster. However, a new clinic may be located in the larger cluster if insufficient information is available to locate the clinic in one of the sub-clusters.

According to various implementations, any of a variety of clustering techniques may be used. These techniques include, but are not limited to: K-means clustering, Fuzzy C-means clustering, Hierarchical clustering, and Mixture of Gaussian clustering.

In some embodiments, each collection of data about a clinic may be treated as a vector in an N-dimensional space. Then, a distance measure may be calculated between any or all pairs of vectors. Finally, pairs of clinics whose vectors have a relatively low distance measure may be grouped into the same cluster. A variety of distance measures may be used, such as for example the Minkowski metric provided by the following formula, where d_{(x,y)} is the distance between patients x and y, n is the number of dimensions in the vector space, i is an index over those dimensions, and p is the order of the metric. In particular embodiments, a value of p=1 or p=2 may be used, rendering the metric a Manhattan distance or a Euclidean distance respectively.

$${d}_{( x , y )} = {( {\sum }_{i}^{n} {\vert {x}_{i} - {y}_{i} \vert}^{p} )}^{\frac{1}{p}}$$

At 514, clinics are assigned to the profile clusters. According to various embodiments, for each clinic, any available information about the clinic may be used to assign the clinic to a cluster. For example, an existing clinic may be associated with one or more of clinic demographic data, practitioner information, clinic geographic information, and clinic practice characteristic data. However, a new clinic may be associated with a more limited selection of data.

In particular embodiments, one or more multilabel classification algorithms may be used to assign clinics to clusters. In such an algorithm, the number of labels may be determined based on the number of clusters identified by the cluster engine. For example, the number of labels may be the same as the number of clusters. The types of cluster assignment procedures that may be used may include, but are not limited to: K-Nearest Neighbor, Logistic Regression, Random Forest, Extremely Randomized Trees, AdaBoost, Gradient Boosting Trees, and Feedforward Neural Network.

At 516, performance characteristics are determined for each cluster. According to various embodiments, the performance characteristics may include any of the information identified at operation 506. Determining cluster-level performance characteristics may include identify statistics such as mean, median, mode, or other measures of central tendency. Alternately, or additionally, determining cluster-level performance characteristics may include identifying statistics such as standard deviation, variance, skewness, kurtosis, or other measures of distributional spread. In particular embodiments, cluster-level performance characteristics may include other types of statistical operations such as the estimation of kernel densities or other distributional attributes of the data.

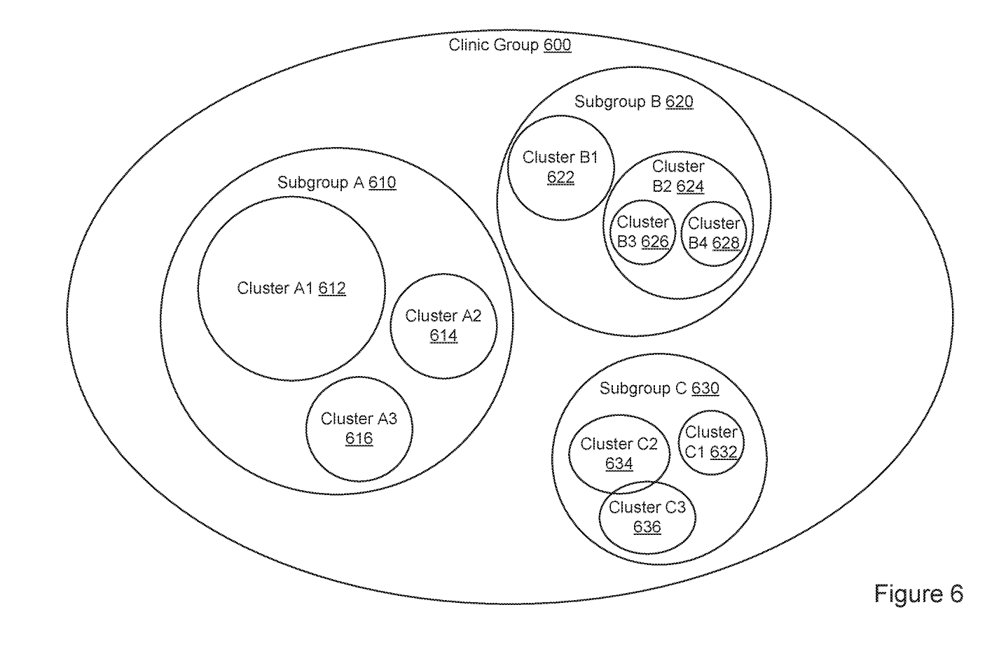

FIG. 6 illustrates one example of an arrangement of medical clinics into clusters. The clinic group 600 includes a number of medical clinics across potentially many different areas. For example, the clinic group 600 may include all clinics known to the clustering system, or may include only a subset of such clinics. The clinics are divided into subgroups 610, 620, and 630 and into clusters 612, 614, 616, 622, 624, 626, 628, 632, 634, and 636.

In particular embodiments, medical clinics included in the clinic analytics system may be divided into subgroups and then clustered within those subgroups. For example, clinics may be divided into subgroups based on characteristics such as geographic location or clinic type. Alternately, clinics may be clustered across the entire system without division into subgroups. The decision as to whether to divide clinics into subgroups may be made at least in part based on whether clinics in different geographic locations or across different clinic types tend to exhibit similar observable characteristics or are similar across other dimensions such as regulatory regimes.

According to various embodiments, clusters may differ along dimensions such as size and number. For example, a subgroup of clinics may initially be divided into a relatively limited number of clusters. However, as more clinics are added to the system and as more data is available for clustering, the number of clusters may be increased in order to refine the analysis of clinic performance characteristics. In some instances, individual clinics may not be members of a cluster. For example, a clinic with data that is relatively dissimilar to that of other clinics may be treated individually or assigned a general default cluster applicable to otherwise unclassified clinics.

In particular embodiments, one or more clusters may be arranged in a hierarchical fashion. For example, cluster B3 626 and cluster B4 628 are both members of the cluster B2 624. Such a situation may arise when clinics naturally cluster along one variable, such as geographic location, while dividing along another variable, such as income.

In particular embodiments, one or more clusters may overlap. That is, a clinic may be a member of two different clusters. For example, cluster C2 634 overlaps with cluster C3 636. As another example, the Subgroup A 610 overlaps with the Subgroup B 620. Such as situation may arise when clinics are on the boundaries of two clusters. For instance, such clinics may be compared with characteristics of either or both cluster depending on the particular performance characteristic being compared.

FIG. 7 illustrates one example of a medical practice analytics method 700 that may be performed in accordance with techniques and mechanisms described herein. According to various embodiments, the method 700 may be performed at a medical practice analytics system such as the system 200 described with respect to FIG. 2. The method 700 may be performed at least in part to analyze the information collected in the method 300 and to provide performance information to one or more medical practice clinics based on that analysis.

As an example of the application of method 7, clinics may be clustered along one or more dimensions such as geographic location, the type of service provided, the number of practitioners, the types of practitioners, the percentage of patients having pre-paid phone contracts, the average distance traveled by patients, the insurance providers associated with patients, or any other suitable dimension. Then, one or more comparisons may be made along dimensions such as procedure pricing, under-charging, patient insurance funds remaining, clinic profitability, practitioner efficiency, or other such outcomes of a designated medical practice clinic compared to other medical practice clinics in the same cluster.

At 702, a request to analyze a medical practice clinic is received. According to various embodiments, the request may be generated periodically, manually, or upon the detection of a triggering event. For example, a medical practice clinic may be analyzed daily, weekly, monthly, or at some other time interval. As another example, an analysis of a medical practice clinic may be generated at the request of a user such as a systems administrator or a user associated with the medical practice clinic. As yet another example, an analysis of a medical practice clinic may be generated when the clinic joins the system or when updated data is added to the system.

At 704, a performance measure is selected for comparison. According to various embodiments, the selection may be made automatically or based on user input. For example, each suitable performance measure may be analyzed in turn until all suitable outcome measures have been analyzed. As another example, a user may manually request to compare a particular performance measure.

In some embodiments, any of a variety of performance measures may be selected. In a first example, clinics may be ranked and/or stacked according to criteria such as the percentage of the clinic's patients who visited the clinic during a designated period of time, the number of patients who failed to visit the clinic during a designated period of time, the average time period between visits for the clinic's patients, the average revenue per practitioner, the average revenue per clinic room or chair, the average revenue per clinic visit per patient appointment, the accounts receivable, the number of days the clinic was open, the average amount of insurance remaining for the clinic's patients, the average percentage of the medical fees covered by patient insurance, the type and profitability of the clinic's patient's insurance providers, patient demographics, profit gained from recall appointment, patient communication performance information, patient satisfaction as measured by quality improvement surveys, the percentage of a clinic's patients having a pre-paid cell phone plan, a clinic's net promoter score, and the comparative performance of a clinic's patient visit planning proportion vs the actual proportion of clinic patient visits according to visit type (e.g., hygiene, treatment, surgical, etc.). In particular embodiments, data can be gathered on patient reviews on online review websites along dimensions such as quality, quantity, and spread across platforms.

At 706, one or more dimensions relevant for the selected performance measure are selected. According to various embodiments, the dimensions may include one or more observable characteristics associated with the clinics. For example, the dimensions may include some or all of the information related to patient demographic information, clinic geographic information, and/or practice characteristics.

In some embodiments, the dimensions may be identified by determining a relationship between particular performance measures and the selected outcome measure. For example, the system may automatically determine that clinics that have similar numbers of practitioners may also tend to have relatively similar levels of staff overhead expenses. As another example, the system may automatically determine that clinics located in the same general geographic area also tend to exhibit similar patient recall rates.

At 708, one or more related medical practice clinics is identified. According to various embodiments, the medical practice clinics may be identified based at least in part on the clusters determined as discussed with respect to the method 500 shown in FIG. 5.

In some embodiments, the related medical practice clinics may be identified irrespective of the selected performance measure. Alternately, the related medical practice clinics may be identified by clustering entirely or predominantly on the dimensions selected at operation 706.

At 710, performance characteristic data is created based on a comparison of the designated medical practice clinic to the related medical practice clinics. According to various embodiments, the performance characteristic data may indicate a performance of the designated medical practice clinic relative to the related medical practice clinics along the designated performance measure. For example, the performance characteristic data may indicate whether and to what degree the designated medical practice clinic is above or below the mean, median, mode, or some other measure of central tendency of the selected performance measure among the related medical practice clinics. For instance, the performance characteristic may indicate information such as a number of procedures per practitioner per day, an amount billed for a designated procedure, or a patient recall rate of the designated medical practice clinic relative to the related medical practice clinics.

In particular embodiments, the performance characteristic data may include information characterizing the variance of the distribution of the performance characteristic among the related medical practice clinics. For example, the performance characteristic data may indicate a number of standard deviations of variation of the performance measure for the designated medical practice clinic from the mean or other measure of central tendency of the performance measure for the related medical practice clinics.

At 712, a determination is made as to whether to select an additional performance measure for comparison. According to various embodiments, the determination may be made automatically or based on user input. For example, each suitable performance measure may be analyzed in turn until all suitable outcome measures have been analyzed. As another example, a user may manually request to compare an additional performance measure.

At 714, a performance characteristic message is transmitted to the medical practice clinic. According to various embodiments, the performance characteristic message may include some or all of the performance characteristic data created at operation 712. Alternately, or additionally, the performance characteristic message may include aggregate data associated with the medical practices identified at operation 708.

In particular embodiments, the performance characteristic message may be transmitted by the data source communication interface, for instance in conjunction with the connector associated with the medical practice clinic. Alternately, or additionally, the performance characteristic message may be transmitted via any suitable medium, which may include, but are not limited to: email, text message, voicemail, and HTTP.

In some embodiments, the performance characteristic message may be transmitted in association with user interaction with a user interface provided via the internet. For instance, a user may authenticate to a front-end user interface associated with the medical practice analytics system, identify one or more performance characteristics for analysis, and receive a response message via the user interface.

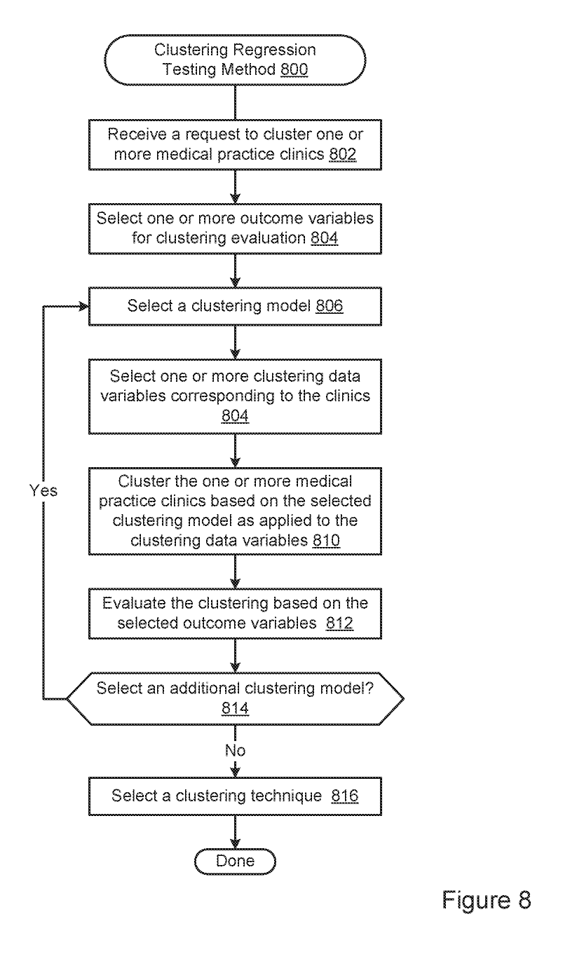

FIG. 8 illustrates one example of a clustering regression testing method 800 that may be performed in accordance with techniques and mechanisms described herein. According to various embodiments, the method 800 may be performed at a clinic analytics system such as the system 202 shown in FIG. 2.

At 802, a request is received to cluster one or more medical practice clinics. According to various embodiments, the request may be generated automatically or manually. For example, clinics may be clustered at the request of a user such as a systems administrator. As another example, clinics may be clustered periodically or at scheduled times, such as once per week or when a substantial amount of new clustering data is received.

At 804, one or more outcome variables is selected for evaluating the clustering procedure. In some implementations, any of a variety of outcome variables may be used. In a first example, one outcome variable may be a known matching of some of the clinics along one or more dimensions. For instance, organizational data may indicate that two clinics are part of the same company and should therefore be grouped within the same cluster. Alternately, manual or alternate automated analysis may indicate that two or more clinics are highly similar and should be located within the same cluster. In this case, the indication that two clinics should be positioned within the same cluster may serve as an outcome variable.

In a second example, any data variable that is not used for the purpose of clustering may serve as an outcome variable. For example, clinics may be clustered on a range of variables, but a variable such as average income among the patients of the clinic may be excluded from the analysis. Then, this average income variable may be treated as an outcome variable for evaluating the performance of the clustering.

In a third example, data observations may be divided according to a particular time threshold into past observations and future observations. In this case, the past observations may be used to divide the medical clinics into clusters, while the future observations may be used to evaluate the performance of the clustering.

In a fourth example, outcome variables may be received from an external source. For instance, the system may receive sophisticated insurance data indicating various measured or predicted characteristics associated with medical practices. Such data may provide a source of truth for the evaluation of clustering models.

In a fifth example, outcome variables may be selected that cover only a subset of the medical practice clinics. For example, clinics may be clustered using data that is commonly available for all clinics. However, the evaluation of the clustering procedure may be performed using data that is only available for some of the clinics. In this way, data availability concerns may be mitigated when establishing clusters while at the same time taking advantage of incomplete but high-quality data. For instance, patient demographic information such as average income may be available for some clinics but not for others.

At 806, a clustering model is selected. According to various implementations, any of a variety of clustering techniques may be used. These techniques include, but are not limited to: K-means clustering, Fuzzy C-means clustering, Hierarchical clustering, density-based clustering, centroid-based clustering, and Mixture of Gaussian clustering.

At 808, one or more clustering data variables corresponding to the clinics is selected. According to various embodiments, clinics may be clustered on any available data. For example, geographic, practitioner, and practice characteristic data may be available for all or virtually all clinics, while patient demographic information may be incomplete for clinics newly added to the system. Thus, clinics may be grouped according to similarity along demographic, practitioner, and practice characteristic dimensions to determine an initial assignment of clusters.

In particular embodiments, the same or different clustering models may be applied to the same or different clustering data variables. For example, particular data variables may be more or less relevant to particular clustering models. As another example, the same clustering model may be applied to different clustering variables in order to determine the relative performance of different clustering variables for clustering.

At 810, the one or more medical practice clinics are clustered based on the selected clustering model as applied to the selected clustering data variables. In particular embodiments, clusters may be determined in a hierarchical fashion. For example, a particular cluster of clinics may include medical practices located within the same geographic region. However, this cluster of clinics may include one group that is associated with lower-income patients and another group that is associated with higher-income patients. In this example, the two groups may be treated as sub-clusters of the larger cluster. However, a new clinic may be located in the larger cluster if insufficient information is available to locate the clinic in one of the sub-clusters.

In some embodiments, each collection of data about a clinic may be treated as a vector in an N-dimensional space. Then, a distance measure may be calculated between any or all pairs of vectors. Finally, pairs of clinics whose vectors have a relatively low distance measure may be grouped into the same cluster. A variety of distance measures may be used, such as for example the Minkowski metric provided by the following formula, where d_{(x,y)} is the distance between patients x and y, n is the number of dimensions in the vector space, i is an index over those dimensions, and p is the order of the metric. In particular embodiments, a value of p=1 or p=2 may be used, rendering the metric a Manhattan distance or a Euclidean distance respectively. Additional details regarding the construction of clusters are discussed with respect to FIG. 5.

At 812, the clustering is evaluated based on the selected outcome variables. According to various embodiments, evaluating the clustering technique may involve determining the extent to which clinics that share the same cluster as assigned by the clustering technique also exhibit commonality in the selected one or more outcome variables. For example, if two clinics are known to share the same characteristics according to outcome variable, then the clustering procedure can be evaluated to determine whether those two clinics were assigned to the same cluster.

In particular embodiments, when evaluating the clustering technique, the clustering technique may be assigned a numerical indicator of quality based on the concordance between the assigned clusters and the outcome variables. Then, the different clustering techniques may be ranked based on the numerical quality indicator.

At 814, a determination is made as to whether to select an additional clustering model. According to various embodiments, each of the available clustering mechanisms or techniques may be selected for application, sequentially or in parallel. In this way, the different clustering mechanisms or techniques may be computed and compared with one another.

At 816, a clustering technique is selected. According to various embodiments, the clustering technique may be selected by determining which of the clustering techniques applied at operation 810 resulted in the best performance as evaluated at operation 812. For example, the numerical quality indicators of different clustering techniques may be compared to identify the highest quality clustering technique.

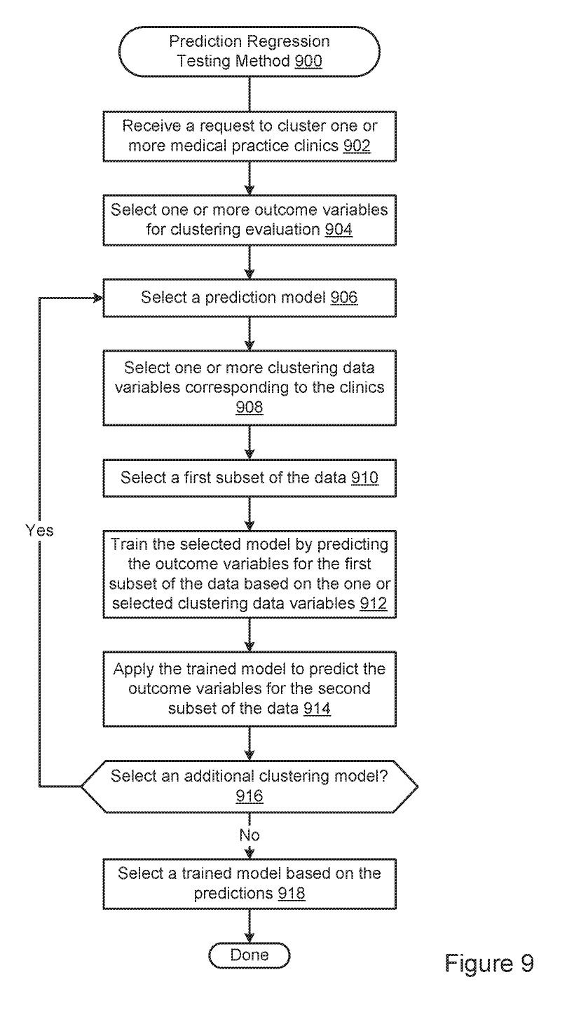

FIG. 9 illustrates one example of a prediction regression testing method 900 that may be performed in accordance with techniques and mechanisms described herein. According to various embodiments, the method 900 may be performed at a clinic analytics system such as the system 202 shown in FIG. 2.

At 902, a request is received to cluster one or more medical practice clinics. According to various embodiments, the request may be generated automatically or manually. For example, clinics may be clustered at the request of a user such as a systems administrator. As another example, clinics may be clustered periodically or at scheduled times, such as once per week or when a substantial amount of new clustering data is received.

At 904, one or more outcome variables is selected for evaluating the clustering procedure. In some implementations, any of a variety of outcome variables may be used. In a first example, one outcome variable may be a known matching of some of the clinics along one or more dimensions. For instance, organizational data may indicate that two clinics are part of the same company and should therefore be grouped within the same cluster. Alternately, manual or alternate automated analysis may indicate that two or more clinics are highly similar and should be located within the same cluster. In this case, the indication that two clinics should be positioned within the same cluster may serve as an outcome variable. In particular embodiments, the selection of an outcome variable at operation 904 may be substantially similar to the operation 804 discussed with respect to FIG. 8.

At 906, a prediction model is selected. According to various embodiments, any of a variety of prediction models may be employed. For example, the types of prediction models that may be used may include, but are not limited to: parametric models, non-parametric models, neural networks, ordinary least squares regression models, non-linear regression models, generalized linear models, support vector machines, semiparametric regression models, and random forest models.

At 908, one or more clustering data variables corresponding to the clinics is selected. According to various embodiments, clinics may be clustered on any available data. For example, geographic, practitioner, and practice characteristic data may be available for all or virtually all clinics, while patient demographic information may be incomplete for clinics newly added to the system. Thus, clinics may be grouped according to similarity along demographic, practitioner, and practice characteristic dimensions to determine an initial assignment of clusters.

In particular embodiments, the same or different clustering models may be applied to the same or different clustering data variables. For example, particular data variables may be more or less relevant to particular clustering models. As another example, the same clustering model may be applied to different clustering variables in order to determine the relative performance of different clustering variables for clustering.

At 910, a first subset of the data is selected. According to various embodiments, the data may be divided into subsets for training and evaluation purposes. For example, 70% of the data may be selected in the first subset for training, while 30% may be reserved for evaluation. According to various embodiments, the proportion of data selected for training purposes may depend on any of various considerations, such as the amount of data available for analysis and the type of model employed for prediction.

At 912, the selected model is trained by predicting the outcome variables for the first subset of the data based on the one or more selected clustering data variables. For example, in the case of a linear regression model, coefficients for each of the parameters may be estimated using the training data. As another example, in the case of a neural network, the neural network may be trained to predict the outcome data using the training data.

At 914, the trained model is applied to predict the outcome variables for the second subset of the data. According to various embodiments, the application of the trained model to the second subset of the data may involve determining predicted values for the clustering data variables associated with the second subset of the data. The predicted values may then be compared with the outcome variables associated with the second subset of the data to determine a prediction quality. For example, in the case of linear regression, a goodness of fit measure such as R squared may be compared. As another example, in the case of a binary outcome variable, a percentage of accurate predictions may be determined.

In particular embodiments, when evaluating the prediction technique, the prediction technique may be assigned a numerical indicator of quality based on the concordance between the predicted outcomes and the actual outcomes for the second subset of the data. Then, the different prediction techniques may be ranked based on the numerical quality indicator.

At 916, a determination is made as to whether to select an additional prediction model. According to various embodiments, each of the available prediction mechanisms or techniques may be selected for application, sequentially or in parallel. In this way, the different prediction mechanisms or techniques may be computed and compared with one another.

At 918, a trained model is selected. According to various embodiments, the prediction technique may be selected by determining which of the prediction techniques applied at operation 911 resulted in the best performance as evaluated at operation 914. For example, the numerical quality indicators of different clustering techniques may be compared to identify the highest quality clustering technique.

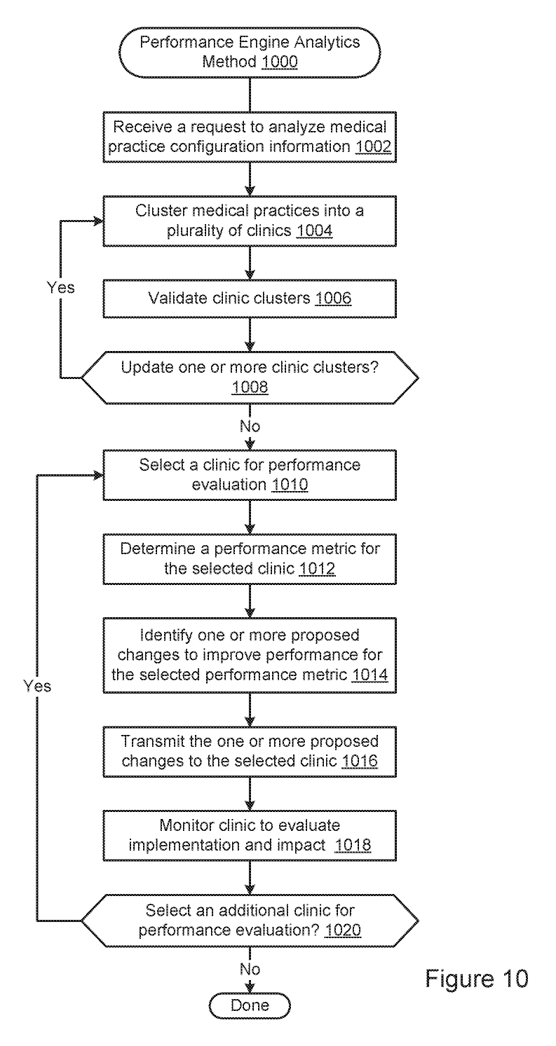

FIG. 10 illustrates one example of a performance engine analytics method 1000 that may be performed in accordance with techniques and mechanisms described herein. According to various embodiments, the method 1000 may be performed at a clinic analytics system such as the system 202 shown in FIG. 2.

At 1002, a request is received to analyze medical practice configuration information for one or more clinics. According to various embodiments, the request may be generated periodically, at scheduled times, or upon the detection of a triggering event. For example, the request may be generated when a new clinic is added to the system, or when a user associated with a clinic requests a trial of the system. As another example, the request may be generated on a daily, weekly, or monthly basis.

At 1004, medical clinics are clustered into a plurality of clinics. Techniques for assigning clinics to clusters are discussed throughout the application, and in particular with respect to the method 500 shown in FIG. 5 and the example configuration of clinics and clusters shown in FIG. 6. As discussed herein, clinics may be clustered based on any number of dimensions. For example, clinics may be clustered based on whether or not they have practice management software installed and, if so, the type of practice management software. As another example, clinics may be clustered on patient and/or practitioner demographics and numbers.

At 1006, the clustering of the clinics is validated based on one or more data sources. In some implementations, the clustering of a clinic may be validated by employing data not used in the initial clustering procedure. For example, data characterizing clinics may be newly received or unavailable during the initial clustering phase. As another example, data characterizing clinics may be only available for some of the clinics and therefore be retained for cluster evaluation rather than cluster specification. As yet another example, clusters may be manually reviewed for accuracy.

At 1008, a determination is made as to whether to update one or more of the clinic clusters. According to various embodiments, the determination may be made at least in part based on the validation information determined at 1006. For example, if the validation procedure indicates that one or more clinics are improperly clustered, then the clinic clusters may be updated before performing further analysis.

At 1010, a clinic measure is selected for performance evaluation. According to various embodiments, the selection may be made automatically or based on user input. For example, an administrator may select particular clinics for performance evaluation. As another example, a clinic may be selected for performance evaluation when the clinic is newly added to the system. As yet another example, each clinic may be analyzed in sequence or in parallel until all suitable clinics have been analyzed.

At 1012, a performance metric is determined for the selected clinic. In some embodiments, the performance metric may be selected manually. For example, a systems administrator or a user associated with the clinic may request that the clinic be analyzed according to a particular performance metric. According to various embodiments, the performance metric may be selected automatically and/or dynamically. For instance, the selected clinic may be compared with other clinics in the same clustered across potentially many different performance metrics. Then, a performance metric may be selected where the clinic falls substantially below the average, highest, or other value for the performance metric as exhibited by other clinics in the same cluster.

The types of performance metrics that may be analyzed may include, but are not limited to: the percentage of the clinic's patients who visited the clinic during a designated period of time, the number of patients who failed to visit the clinic during a designated period of time, the average time period between visits for the clinic's patients, the average revenue per practitioner, the average revenue per clinic room or chair, the average revenue per clinic visit per patient appointment, the accounts receivable, the number of days the clinic was open, the average amount of insurance remaining for the clinic's patients, the average percentage of the medical fees covered by patient insurance, the type and profitability of the clinic's patient's insurance providers, patient demographics, profit gained from recall appointment, patient communication performance information, patient satisfaction as measured by quality improvement surveys, the percentage of a clinic's patients having a pre-paid cell phone plan, a clinic's net promoter score, and the comparative performance of a clinic's patient visit planning proportion vs the actual proportion of clinic patient visits according to visit type (e.g., hygiene, treatment, surgical, etc.).

At 1014, one or more proposed changes are identified to improve performance of the clinic for the selected performance metric. According to various embodiments, the types changes that may be proposed may include, but are not limited to: a patient recall schedule, an amount charged for a particular procedure, a patient communication schedule, a visit reminder procedure, incentives or suggestions to improve Quality Improvement (QI) survey response rate, content or copy of the patient communication messages, the medium for communication (text, email, phone) to facilitate various features within the application (QI Surveys, Social Review Requests, Patient referrals, etc.), the number of practitioners engaged to optimize treatment time, the number of practitioners engaged to optimize patient satisfaction, the optimal in-office techniques leading to high patient satisfaction, and/or the treatments often missed or skipped that are covered by patients insurance and if changed would be likely to yield a predicted higher revenue amount. In some embodiments, proposed changes may be identified based on any of a number of analytical tools.

In a first example, the highest performing members of the cluster along the selected performance metric may be compared with the selected clinic. Then, if the highest performing members tend to adopt a particular practice that is not adopted by the selected clinic, the selected clinic may be provided with a recommendation to adopt the particular practice.

In a second example, the performance engine may implement a prediction algorithm that successively and virtually alters various practice data associated the selected clinic and predicts a change in the selected performance metric based on the performance and characteristics of other clinics within the same cluster. Then, the change that results in a substantial predicted improvement in the selected performance metric may be provided to the selected clinic as a recommendation.

At 1016, the one or more proposed changes are transmitted to the selected clinic. In some embodiments, the clinic may include a computer system that has installed thereon an application configured to interact with the performance engine. Such an application may be configured for the secure transmission of patient data, performance metrics, and other such data between the performance engine and the clinic. When such a system is available, the proposed change may be transmitted via an alert message. In particular embodiments, the proposed change may be transmitted along with an estimate of a time commitment associated with making the proposed change (e.g., 7 minutes, 3 minutes, 2 hours, etc.).

In particular embodiments, the performance engine may communicate with the clinic via a message medium such as email. For example, the clinic may be in a trial phase in which the clinic does not yet have a computer system on which an application is installed that facilitates direct communication between the clinic and the performance engine.

At 1018, the clinic is monitored to evaluate implementation and impact of the proposed changes. In some implementations, monitoring the clinic may involve communication conducted via an application installed at a computing system associated with the clinic. For example, the clinic's patient management system may communicate with the performance engine to indicate whether the proposed change has been implemented. As another example, the clinic's patient management system may communicate updated performance information, which may reflect changes in performance caused by the implementation of the proposed change. The performance information may then be used to update the prediction process implemented at operation 1014.

At 1020, a determination is made as to whether to select an additional clinic for performance evaluation. According to various embodiments, additional clinics may be selected until all clinics are analyzed. As discussed with respect to operation 1010, clinics may be identified for performance analysis periodically, upon detection of a triggering event, or upon request.

In particular embodiments, a clinic may be analyzed multiple times. For example, a clinic may first be evaluated for performance and supplied with one or more proposed changes. Then, at a later point in time, the performance of the clinic may be detected and evaluated to determine the impact of the proposed changes. For instance, the implementation of a proposed change may require some amount of time, or the proposed change may require time to impact the performance metric after implementation.

In the foregoing specification, the invention has been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

1. A system comprising:
a clinic information database implemented on one or more storage devices, the clinic information database storing clinic data characterizing each of a plurality of medical practice clinics, the clinic data including medical practice data indicating one or more medical practice characteristics of the respective medical practice clinic;
a clinic data cluster engine implemented on a processor, the clinic data cluster engine operable to determine a respective plurality of clinic clusters based on the clinic information for each of a plurality of clustering mechanisms, each clinic cluster including a respective subset of the plurality of clinic, the respective subset of the plurality of clinics sharing similar clinic information; and
a clinic data analytics engine configured to evaluate the performance of each of the plurality of clustering mechanisms to produce a respective performance evaluation by determining a respective predicted outcome variable for each of the respective clustering mechanisms and each of the respective clinic clusters and comparing each of the respective predicted outcome variable with a respective observed outcome variable.

2. The system recited in claim 1, wherein the cluster data analytics engine is further configured to:
select a designated one of the clustering mechanisms based on the performance evaluations; and
apply the designated clustering mechanism to predict a designated outcome variable for a designated one of the medical practice clinics.

3. The system recited in claim 2, wherein applying the designated clustering mechanism involves:
identifying an actual value for the designated outcome variable for the designated medical practice clinic;
determining a proposed policy change for the medical practice clinic; and
predicting a second value for the designated outcome variable based on the proposed policy change.

4. The system recited in claim 3, the method further comprising:
electronically transmitting an indication of the proposed policy change and the predicted second value for the designated outcome variable to a computing device associated with the medical practice clinic.

5. The system recited in claim 1, wherein the clinic data clustering engine is operable to determine the plurality of clinic clusters via a mechanism selected from the group consisting of: centroid k-means clustering, density-based spatial clustering, connectivity-based clustering, and distribution-based clustering.

6. The system recited in claim 1, wherein the clinic data clustering engine is configured to assign the plurality of clinics to the plurality of clusters via a mechanism selected from the group consisting of: K-Nearest Neighbor, Logistic Regression, Random Forest, Extremely Randomized Trees, AdaBoost, Gradient Boosting Trees, Feedforward Neural Network.

7. the system recited in claim 1, the system further comprising:
a data source communication interface that includes a plurality of clinic data connectors, each of the clinic data connectors configured to retrieve clinic data from a respective clinic data storage system via a respective application procedure interface, each of the clinic data storage systems storing information associated with a respective medical practice clinic, the retrieved clinic data including performance data indicating one or more performance characteristics of the respective medical practice clinic.

8. The system recited in claim 1, wherein the medical practice data includes patient demographics data, the patient demographics data identifying aggregate characteristics of one or more patients associated with the respective medical practice clinic.

9. The system recited in claim 1, wherein the medical practice data includes geographic data, the geographic data identifying or characterizing a geographic locale associated with the respective medical practice clinic.

10. The system recited in claim 1, wherein the medical practice data includes medical practice information selected from the group consisting of: a number of medical practitioners associated with the clinic, one or more types of medical practitioners associated with the clinic, one or more types of medical procedures performed at the clinic, and one or more medical specialties associated with the clinic.

11. A method comprising:
retrieving clinic data from a clinic information database implemented on one or more storage devices, the clinic data characterizing each of a plurality of medical practice clinics, the clinic data including medical practice data indicating one or more medical practice characteristics of the respective medical practice clinic;
at a clinic data cluster engine implemented on a processor, determining a respective plurality of clinic clusters based on the clinic information for each of a plurality of clustering mechanisms, each clinic cluster including a respective subset of the plurality of clinic, the respective subset of the plurality of clinics sharing similar clinic information; and
at a clinic data analytics engine, evaluating the performance of each of the plurality of clustering mechanisms to produce a respective performance evaluation by determining a respective predicted outcome variable for each of the respective clustering mechanisms and each of the respective clinic clusters and comparing each of the respective predicted outcome variable with a respective observed outcome variable.

12. The method recited in claim 11, the method further comprising:
selecting a designated one of the clustering mechanisms based on the performance evaluations; and
applying the designated clustering mechanism to predict a designated outcome variable for a designated one of the medical practice clinics.

13. The method recited in claim 12, wherein applying the designated clustering mechanism involves:
identifying an actual value for the designated outcome variable for the designated medical practice clinic;
determining a proposed policy change for the medical practice clinic; and
predicting a second value for the designated outcome variable based on the proposed policy change.

14. The method recited in claim 13, the method further comprising:
electronically transmitting an indication of the proposed policy change and the predicted second value for the designated outcome variable to a computing device associated with the medical practice clinic.

15. The method recited in claim 11, wherein the clinic data clustering engine is operable to determine the plurality of clinic clusters via a mechanism selected from the group consisting of: centroid k-means clustering, density-based spatial clustering, connectivity-based clustering, distribution-based clustering.

16. The method recited in claim 11, wherein the clinic data clustering engine is configured to assign the plurality of clinics to the plurality of clusters via a mechanism selected from the group consisting of: K-Nearest Neighbor, Logistic Regression, Random Forest, Extremely Randomized Trees, AdaBoost, Gradient Boosting Trees, Feedforward Neural Network.

17. The method recited in claim 11, the system further comprising:
a data source communication interface that includes a plurality of clinic data connectors, each of the clinic data connectors configured to retrieve clinic data from a respective clinic data storage system via a respective application procedure interface, each of the clinic data storage systems storing information associated with a respective medical practice clinic, the retrieved clinic data including performance data indicating one or more performance characteristics of the respective medical practice clinic.

18. The method recited in claim 11, wherein the medical practice data includes patient demographics data, the patient demographics data identifying aggregate characteristics of one or more patients associated with the respective medical practice clinic.

19. The method recited in claim 11, wherein the medical practice data includes geographic data, the geographic data identifying or characterizing a geographic locale associated with the respective medical practice clinic.

20. One or more computer readable media having instructions stored thereon for performing a method, the method comprising:
retrieving clinic data from a clinic information database implemented on one or more storage devices, the clinic data characterizing each of a plurality of medical practice clinics, the clinic data including medical practice data indicating one or more medical practice characteristics of the respective medical practice clinic;
at a clinic data cluster engine implemented on a processor, determining a respective plurality of clinic clusters based on the clinic information for each of a plurality of clustering mechanisms, each clinic cluster including a respective subset of the plurality of clinic, the respective subset of the plurality of clinics sharing similar clinic information; and
at a clinic data analytics engine, evaluating the performance of each of the plurality of clustering mechanisms to produce a respective performance evaluation by determining a respective predicted outcome variable for each of the respective clustering mechanisms and each of the respective clinic clusters and comparing each of the respective predicted outcome variable with a respective observed outcome variable.
